(19) United States
(12) Patent Application Publication
(10) Pub. No.: US 2024/0225965 A1
(43) Pub. Date: Jul. 11, 2024

(54) Lipstick Use And Application

(71) Applicant: Kinky Kitty, LLC, Cypress, TX (US)

(72) Inventor: Kimberly Hoof, Cypress, TX (US)

(73) Assignee: Kinky Kitty, LLC, Cypress, TX (US)

(21) Appl. No.: 18/407,573

(22) Filed: Jan. 9, 2024

Related U.S. Application Data
(60) Provisional application No. 63/437,888, filed on Jan. 9, 2023.

(51) Int. Cl. A61K 8/04; A61Q 1/06

(52) U.S. Cl. CPC A61K 8/04; A61Q 1/06; A61K 2800/87

(57) ABSTRACT

A method for rotating a base of a lipstick device to move an emulsion structure and applying the emulsion structure to a tissue area.

### CROSS REFERENCE TO RELATED APPLICATIONS

This application claims the priority of U.S. Provisional Patent Application No. 63/437,888, filed Jan. 9, 2023, which is incorporated by reference in its entirety.

### BACKGROUND

Cosmetics and other beauty products are often employed for such grooming and styling purposes. Such beauty products include mascara, kohl, lip balm, lipstick and so forth; the beauty products enable facial defining features that are regarded as being aesthetically attractive to be visually emphasized. The beauty products, for example lipstick and lip balm, often come in a form of a pallet, a crayon, a bullet and the like. When the lip balms and lipsticks are provided in a form of an emulsion structure, they are often provided in a casing that stores the emulsion structure.

### BRIEF DESCRIPTION OF THE DRAWINGS

These drawings illustrate certain aspects of the presented disclosure and should not be used to limit or define the disclosure.

FIG. 1 illustrates a lipstick device.

### DETAILED DESCRIPTION

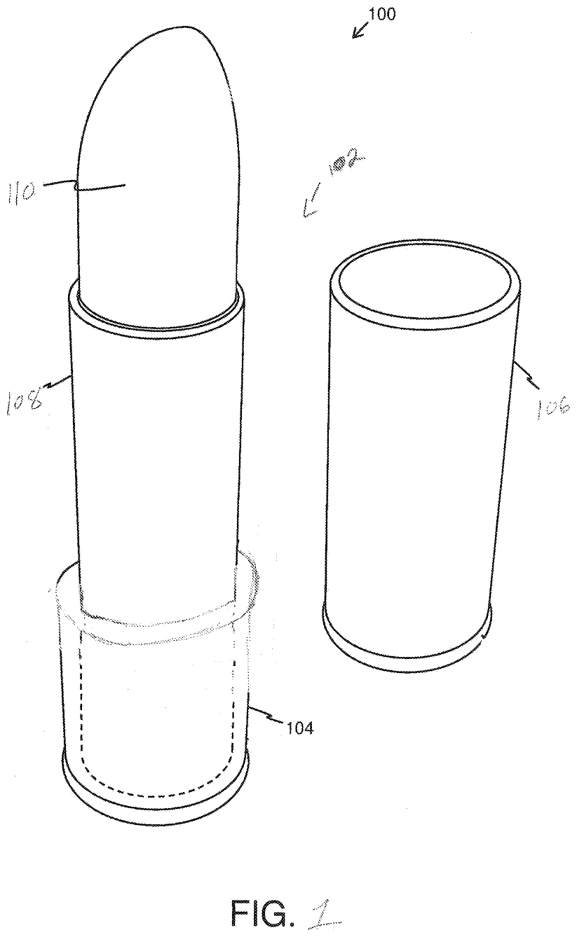

The present disclosure is directed to a method of application for a lipstick device. FIG. 1 illustrates a lipstick device 100. Lipstick device 100 may comprise a casing 102 that may comprise a base 104 and a top 106. In examples, base 104 and top 106 may be mutually coupled through any type of connection. Additionally, base 104 may rotate relative to container 108 about the longitudinal axis of container 108. Housed within container 108 may be emulsion structure 110 that may connect to base 104. During operations, rotating base 104 may move emulsion structure 110 in and/or out of container 108.

Emulsion structure 110 may be an elongate bar, stick and the like, wherein a distal end of the elongate bar, stick and/or the like is tapered. In examples, emulsion structure 110 is manufactured to have a color akin to a lip color, a lip balm, a lip gloss and the like. Further, emulsion structure 110 provides, when applied, a glossy, lustrous, matte, shimmery and the like visual effect. In an example, emulsion structure 110 may comprise one or more tints, a glitter, a fragrance. The lipstick bullet may have an angled, sloped portion on the distal end thereof, that provides a better interface for applying color or balm at corners and sides of lips of the given user. Additionally, emulsion structure 110 may have its distal end terminating in a pointed region. In another example, emulsion structure 110 may have a blunt top, for example a truncated top. emulsion structure 110 may comprise one or more of a non-ionic alkyl phosphate, a base, a humectant, water, a structuring agent, an oil, a pigment, and a glitter.

In other examples, emulsion structure 110 may be odorless (namely, fragrance-free) formula or comprise fragrance, and flight in weight. Additionally, emulsion structure 110 may provide coverage on a desired area of user in a single swipe application of emulsion structure 110. Moreover, emulsion structure 110 when applied to user may have a hydrating and moisturized benefit. Moreover, emulsion structure 110 may have a formula with sun protection and vitamins. Additionally, emulsion structure 110 when applied to a user may provide a satin finish and provides for creamy gliding when swiping.

Emulsion structure 110 is a cosmetic composition comprising at least one of following ingredients: a non-ionic alkyl phosphate; a base; a humectant; a water; a structuring agent; an oil; a pigment; a glitter. Additionally, the cosmetic composition also optionally contains beeswax, ozocerite and candelilla wax. Furthermore, various oils and fats such as at least one of olive oil, mineral oil, cocoa butter, lanolin, synthetic oils, jojoba oil, avocado oil, mango seed butter, fruit oil, natural plant oils, fatty esters, petroleum and so forth may be disposed within emulsion structure 110. Additionally, the cosmetic composition also optionally contains preservatives, antioxidants, fragrance elements, scenting agents, scented oils, vitamins, perfumes, colognes, colorants, pigments, dyes, coloring agents, color additives, sun blocking agents, sunscreen, the like, and/or combinations thereof.

For example, the cosmetic composition may include parabens, phenoxyethanol, vitamin E, BHA, BHT, pentaerythrityl tetraisostearate, isostearyl isostearate, polybutene, oxtyldodecanol, polyglyceryl-3 diisostearate, mica, polyethylene, diisostearyl malate, octydodecyl stearoyl stearate, polybutene, microcrystalline wax, synthetic beeswax, perfumes, silica, tocopheryl acetate, butters, glycerides, hydrogenated glycerides, tin oxide, titanium dioxide, iron oxide, caprylic triglyceride, capric triglyceride, trioctylododecyl citrate, oxtyldodecanol, ozokerite, bis-diglyceryl polyacyladipate-2, polydecene, polyethylene, cholesterol, tribehenin, linoleic acid, sodium hyaluronate, squalene, tocopheryl acetate, phystosterol, sorbitan, isostearate, ceramide, parfum, carmine, saccharin, polmitoyl tripeptide-1, ceramide, pentaerythrityl tetra-di-t-butyl hydroxyhydrocinnamate, trioctyldodecyl citrate, ethylhexyl palmitate, oxtyldodecyl neopentanoate, pentaerythrityl tetraisostearate, lanosterol esters, bis-diglyceryl polyacyladipate-2, synthetic fluorphlogopite, calcium sodium borosilicate, tocopheryl acetate, ascorbyl palmitate, aloe barbadensis leaf extract, serica, pentaerythrityl tetraethylhexanoate, ethylene copolymer, propylene copolymer, palmitic acid, polyisobutene, benzoic acid, barley extract, wheat germ extract, soy bean seed extract, oxybenzone, octinoxate, octisalate, avobenzone, homosalate, the like, and/or combinations thereof.

A chemical composition of cosmetic composition may be tuned so that, for example, a melting temperature of emulsion structure 110 is sufficiently high to withstand heat applied thereto. For example, a melting point of emulsion structure 110 may be higher than a body temperature, higher than 100° F., higher than 110° F., higher than 120° F., higher than 130° F., or higher than 140° F. Additionally, chemical composition of cosmetic composition may be tuned such that emulsion structure 110 is thermally conductive and operable to conduct heat to tissue area. Additionally, chemical composition of cosmetic composition may be tuned such that emulsion structure 110 is imbued with structural stability and operable to resist deformation when exposed to an applied force and/or pressure.

The constituents of emulsion structure 110 provide it with an attractive and long-lasting texture when applied on a given tissue area. The constituents of emulsion structure 110 also provide biological nourishment to the given tissue area. Tissue area may comprise labium superius oris, labium inferius oris, labia minora, labia majora, and/or combinations thereof. Tissue area may additionally, or alternatively, comprise a penis, base, shaft, glans, foreskin, frenulum, external urethral opening, corona of glans, neck of glans, opening of preputial gland, skin, and/or combinations thereof. It will be appreciated that a formula, texture, property associated with emulsion structure 110 is only elucidated in the foregoing for a sake of clarity and is not to be constructed as limiting a variation of implementation of the lipstick bullet.

The cosmetic composition may in some examples, optionally include a cooling agent. Cooling agent may function, for example, by way of evaporative cooling. Suitable compositions capable of undergoing evaporative cooling may comprise, in certain example, one or more compounds having an overall vapor pressure greater than an atmospheric pressure at standard temperature and pressure. Examples of suitable cooling agents may include, for example, menthol.

It is contemplated that a cooling agent may additionally or alternatively comprise, for example, an alcohol, such as one or more alcohols selected from a simple alcohol, an alcohol having a low acute toxicity, ethanol, isopropyl alcohol, glycerol, and combinations thereof. An alcohol may comprise one or more hydroxyl groups. Hydrogen peroxide may be included in the cooling agent to provide a sanitization effect. When used in the emulsion structure 110, it is preferred that the cooling agent have little to no acute toxicity, or otherwise be included in the cosmetic composition in a concentration such that the cosmetic composition has little to no toxicity. For example, any one or more components may be included in the cosmetic composition in an amount of about 1 milligram, 5 milligrams, 10 milligrams, 50 milligrams, 100 milligrams or less per unit volume of the cosmetic composition. A unit volume may comprise the emulsion structure 110, or alternatively, a cubic volume of the cosmetic composition, such as 1 milliliter, 5 milliliters, 10 milliliters, or a liter. In some examples, one or more components of the cosmetic composition, the cosmetic composition, or both may have an EPA toxicity rating of III, and in some examples, IV. In some examples, one or more components of the cosmetic composition, the cosmetic composition, or both may have an oral LD₅₀ of greater than 500 milligrams, greater than 5,000 milligrams, and in some examples, greater than 5,000 milligrams per kilogram of body weight.

In examples wherein one or more cooling agents is included in the cosmetic composition, the emulsion structure 110 may be directly or indirectly applied to tissue area such that a surface temperature of tissue area may drop by at least 0.5 degrees, at least 1 degree C, at least 2 degrees C, at least 3 degrees C, at least 4 degrees C, at least 5 degrees C, or more upon, for example, open-air exposure and/or ventilation for a period of time. A period of time may comprise, for example, 1 second, 2 seconds, 5 seconds, 30 seconds, a minute, 5 minutes, 10 minutes, or 30 minutes.

The cosmetic composition may also, in certain examples, comprise one or more luminescent and/or glow-in-the-dark compounds, such as Zinc Sulfide and Strontium Aluminate, phosphors, phosphorescent compounds, bioluminescent compounds, or the like. In such examples, the cosmetic composition may be applied as a luminescent residue such that chemical energy stored within cosmetic composition is slowly emitted as light to provide a luminescent effect.

Lipstick device 100 may in certain examples, include a heating element (not shown). One or more constituents of a heating element may be disposed in any suitable location in or on lipstick device 100. For example, one or more constituents of heating element may be disposed within emulsion structure 110, within container 108, within base 104, in a region disposed between emulsion structure 110 and base 104, between emulsion structure 110 and container 108, between container 108 and base 104, on an outer surface of container 108, or any combination thereof. Heating element may comprise any suitable device operable to apply heat to emulsion structure 110 and/or tissue area. For example, one or more constituents of heating element may comprise a power source and a resistor. Resistor may be operable to convert power from a power source to thermal energy and generate heat. Power source may comprise one or more batteries and may be rechargeable via one or more input ports (not shown). Heat generated by resistor may be conducted through any constituent of lipstick device 100, such as through emulsion structure 110, container 108, base 104, lid 106, or any combination thereof. Conversely, one or more constituents of lipstick device 100 may act as a thermal insulator to ensure that heat generated by resistor is conducted primarily to a target location through thermally conductive constituents only. A target location may include a region of emulsion structure 110, such as a distal end of emulsion structure 110. One or more thermally conductive components, such as a thermally conductive gel, may be disposed between one of the one or more constituents of heating element and one or more constituents of lipstick device 100. Thermally conductive components may be operable, for example, to minimize contact resistance between heat-conducting constituents, maximize heat transfer, and/or prevent overheating of lipstick device 100.

In some examples, lipstick device 100 may include a housing (not shown). Housing may be connected to, or form a part of, container 108. Alternatively, housing may be simply connected to, or form a part of, lipstick device 110, such as by being connected to a distal end thereof. Housing may have an outlet of any shape or size (not shown) disposed therein. Housing may be operable to allow emulsion structure 110 to extend and protrude through the outlet upon, for example, rotating base 104 relative to container 108. Housing may comprise any suitable shape and may provide a structural and/or an aesthetic function to lipstick device 110. For example, housing may encase at least a portion of emulsion structure 110 to preserve a shape of emulsion structure 110 and/or protect cosmetic composition from exposure to outside contamination. Housing may comprise any suitable material including, for example, silicone, plastic, rubber, and any combination thereof.

The preceding description provides various examples of the systems and methods of use disclosed herein which may contain different method steps and alternative combinations of components. It should be understood that, although individual examples may be discussed herein, the present disclosure covers all combinations of the disclosed examples, including, without limitation, the different component combinations, method step combinations, and properties of the system. It should be understood that the compositions and methods are described in terms of “comprising,” “containing,” or “including” various components or steps, the compositions and methods may also “consist essentially of” or “consist of” the various components and steps. Moreover, the indefinite articles “a” or “an,” as used in the claims, are defined herein to mean one or more than one of the element that it introduces.

All numerical values within the detailed description and the claims herein modified by “about” or “approximately” with respect to the indicated value is intended to take into account experimental error and variations that would be expected by a person having ordinary skill in the art.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values even if not explicitly recited. Thus, every point or individual value may serve as its own lower or upper limit combined with any other point or individual value or any other lower or upper limit, to recite a range not explicitly recited.

Therefore, the present examples are well adapted to attain the ends and advantages mentioned as well as those that are inherent therein. The particular examples disclosed above are illustrative only, and may be modified and practiced in different but equivalent manners apparent to those skilled in the art having the benefit of the teachings herein. Although individual examples are discussed, the disclosure covers all combinations of all of the examples. Furthermore, no limitations are intended to the details of construction or design herein shown, other than as described in the claims below. Also, the terms in the claims have their plain, ordinary meaning unless otherwise explicitly and clearly defined by the patentee. It is therefore evident that the particular illustrative examples disclosed above may be altered or modified and all such variations are considered within the scope and spirit of those examples. If there is any conflict in the usages of a word or term in this specification and one or more patent(s) or other documents that may be incorporated herein by reference, the definitions that are consistent with this specification should be adopted.

What is claimed is:

1. A method comprising:
rotating a base of a lipstick device to move an emulsion structure; and
applying the emulsion structure to a tissue area.
